(19) United States
(12) Patent Application Publication
(10) Pub. No.: US 2019/0022145 A1
(43) Pub. Date: Jan. 24, 2019

(54) CELLULAR BLEND FOR THE REGENERATION OF CHONDROCYTES OR CARTILAGE TYPE CELLS

(71) Applicant: SpinalCyte, LLC, Houston, TX (US)

(72) Inventor: Pete O'Heeron, Houston, TX (US)

(73) Assignee: SpinalCyte, LLC, Houston, TX (US)

(21) Appl. No.: 16/068,096

(22) PCT Filed: Jan. 13, 2017
(86) PCT No.: PCT/US17/13449

§ 371 Date: Jul. 3, 2018

Related U.S. Application Data
(60) Provisional application No. 62/413,587, filed on Oct. 27, 2016.
(60) Provisional application No. 62/278,635, filed on Jan. 14, 2016.

(51) Int. Cl. A61K 35/32; A61K 35/16; A61K 35/19; A61K 35/33; A61K 35/35

(52) U.S. Cl. CPC A61K 35/32; A61K 35/16; A61K 35/35; A61K 35/33; A61K 35/19

(57) ABSTRACT

The present disclosure concerns at least methods and compositions for repairing and/or regenerating a disc of an individual in need thereof. In certain embodiments, an individual that is known to have or suspected to have or at risk for having a degenerated disc is provided a therapeutically effective amount of nucleus pulposus cells, one or more components from the nucleus pulposus, conditioned medium generated from nucleus pulposus cells, and additionally may also be provided fibroblasts, platelet rich plasma (PRP), SOX9 (protein or nucleic acid), and/or Tie2+ cells, for example.

This application claims priority to U.S. Provisional Patent Application Ser. No. 62/278,635, filed Jan. 14, 2016, and to U.S. Provisional Patent Application Ser. No. 62/413,587, filed Oct. 27, 2016, both of which applications are incorporated by reference herein in their entirety.

### TECHNICAL FIELD

The present invention generally concerns at least the fields of medicine, surgery, anatomy, biology, cell biology, and/or molecular biology. In particular aspects, the present invention concerns the fields of spinal disc repair. More particularly, the field of the invention concerns using a cell therapy for the regeneration of chondrocytes or other cartilage type cells.

### BACKGROUND

Typically, cartilage is a tissue that is not naturally regenerated once damaged. Recently, efforts have been made to reconstruct damaged biological tissues by regenerating a portion of the damaged tissues in laboratories. This approach, defined as “tissue engineering,” has raised tremendous attention.

Tissue engineering involves the development of biocompatible materials capable of specifically interacting with biological tissues to produce functional tissue equivalents. Tissue engineering has a basic concept of collecting a desired tissue from an individual, isolating cells from the tissue specimen, proliferating cells, and re-introducing those cells back into the same individual or a different individual. In other aspects, gene therapy capable of attracting or generating the desired cells in vivo is utilized.

Fibroblast cells have been used for the regeneration of chondrocytes or cartilage type cells. The fibroblasts have been proven to induce their differentiation into chondrocytes in a mechanically stressed, hypoxic environment, for example. However, there is no current scientific evidence to support the use of additional cell types to this mixture.

### Disc Degeneration

More than 65 million Americans suffer from lower back pain annually. By age 50, 85% of the population will show evidence of disc degeneration. Degeneration of the intervertebral disc, which is often called degenerative disc disease (DDD) or osteoarthritis of the spine, is a common disorder of the lower spine. Disc degeneration can lead to disorders such as lumbar spinal stenosis (narrowing of the spinal canal that houses the spinal cord and nerve roots), spondylolisthesis (forward slippage of the disc and vertebra), and retrolisthesis (backward slippage of the disc and vertebra). DDD is a degenerative condition that can be painful and greatly affect the quality of life.

Aging is the most common cause of disc degeneration. As the body ages, the discs in the spine lose important cells that make the disc viable, dehydrate, or dry out, and lose their ability to act as shock absorbers between the vertebra. The bones and ligaments that make up the spine also become less flexible and thicken. Unlike muscles, there is minimal blood supply to the discs, so they lack the ability to heal or repair themselves. DDD can result in chronic low back pain that sometimes radiates to the hips, or there is an aching pain in the buttocks or thighs while walking, for example.

It is not clear why some degenerative discs are painful and some are not. Some people have nerve endings that penetrate more deeply into the annulus fibrosus, or outer layer of the disc, than others, making the disc more susceptible to becoming a source of pain. Pain that radiates down the leg, known as sciatica or lumbago, is the result of the nerve root encountering the inner disc material, or the nucleus pulposus, an inflammatory substance that also puts pressure on the nerve. These conditions can cause symptoms such as severe leg pain, difficulty standing and walking, and weakness or numbness in the legs. DDD can lead to a chronic debilitating condition and can have a serious negative impact on a person's quality of life. When DDD is severe, traditional non-operative treatment is often ineffective.

It is challenging to restore full tissue function in damaged or diseased spinal discs. Although there are traditional methods like artificial disc replacements or nucleus substitute, there still exist many shortcomings associated with these therapies. Artificial disc replacements may be prone to dislodgement and transference of mechanical stress to other vertebra above or below the affected area, known as a cascading effect, for example

The present disclosure concerns efficient and simple methods and compositions to successfully repair and/or regenerate spinal disc, for example.

### SUMMARY OF THE INVENTION

Embodiments of the disclosure concern methods and compositions for repair of a joint in a mammalian individual, such as a human, dog, cat, or horse, for example. Although the joint may be of any kind, in specific embodiments the joint is a spinal disc, although multiple spinal discs may be treated at the same or different times in a mammalian individual. Methods and compositions of the disclosure utilize one or more nucleus pulposus components, such as notochordal cells, small chondrocyte-like cells, collagen (such as type II collagen and/or collagen types I, V, VI, IX and XII) fibrils, and/or proteoglycans (such as aggrecan), for example, for repair and/or regeneration of tissue or other matter in a joint.

The present disclosure concerns a therapeutic delivery (for example, by injection or open application via surgical site) of allogeneic, autologous, or xenogeneic cells (such as nucleus pulposus cells), and/or conditioned medium therefrom, to a joint (including a spinal disc) of a mammal in need thereof. In some embodiments, the nucleus pulposus cells (in addition to, or alternatively to, cells that can differentiate into nucleus pulposus cells) are provided with one or more other therapeutic agents. Although the one or more other therapeutic agents may be of any kind, in specific embodiments the agent(s) is a cell, protein, nucleic acid (including a coding sequence, miRNA, mRNA, DNA, shRNA, siRNA, a combination thereof, and the like), small molecule, or combination thereof. The one or more other therapeutic agents may or may not be a component from the nucleus pulposus.

In specific embodiments, a therapeutic agent to be provided to the joint (in addition to nucleus pulposus cells or one or more components from nucleus pulposus) is a small molecule, an expressible nucleic acid, a peptide, a protein, or a combination thereof. Although a variety of nucleic acid(s), peptide(s), and/or protein(s) may be provided with the cells, in specific embodiments the nucleic acid(s), peptide(s), or protein(s) comprise Sox9 and/or platelet-rich plasma (PRP) and/or other nucleic acid(s), peptide(s), or protein(s) that aid in the repair and/or regeneration of cartilage. In specific embodiments, the nucleus pulposus cells and/or cells that can differentiate into nucleus pulposus cells are provided in addition to one or more components of the NP (such as notochordal cells and/or Tie2+cells), including with or without a nutrient matrix or vessel.

In certain embodiments, the present disclosure relates to methods and compositions for biological repair of cartilage (such as in a joint), for example in the spinal disc or other cartilage, using delivery of at least one cartilage-forming mixture. This therapy can act as an in vivo workstation for cartilage restoration, in specific embodiments. In some cases, one or more of the cells and/or other therapeutic agent(s) are provided with an inert device. Although the inert device may be of any kind, in specific embodiments, the inert device comprises a structure that comprises two generally concentric inflatable membranes. One or more of the cells and/or other therapeutic agent(s) may or may not be provided to an individual with a scaffold.

The present disclosure encompasses methods for improving the condition of an aged disc in an individual by providing an effective amount of a cell blend from a healthy disc. Also included are methods for improving the condition of an aged disc in an individual by providing an effective amount of a cell blend one might find in a younger and more healthy disc. Methods of the disclosure include those in which one improves the condition of an aged disc in an individual by providing to the individual with the aged disc an effective amount of a cell blend that one would find in a disc of a person without the onset of the degenerative process. One embodiment of the disclosure includes a method of improving the condition of an aged disc in an individual by providing to the individual with the aged disc an effective amount of a cell blend from, or that one would be found in, one or more discs of one or more persons without the onset of the degenerative process. A cell blend that would be found in one or more discs of one or more persons without the onset of the degenerative process may include an effective amount of nucleus pulpous cells, chondrocytes, fibroblasts (including at least dermal fibroblasts or fibroblasts from connective tissue in the body, including bone, cartilage and/or muscle), stem cells, and/or adipocytes. The cell blend may also comprise non-cellular components, such as collagen fibrils, proteoglycans, and/or aggrecan, for example.

As referred to herein, the onset of disc degeneration may include one or more symptoms of pain and in some cases radiating weakness or numbness stemming from a degenerated disc in the spine and/or disc degeneration may include the breakdown of one or more spinal discs that may include the loss of fluid in the disc and/or tiny tears or cracks in the outer layer (annulus or capsule) of the disc (and, in some cases, the nucleus inside the disc is forced out through the tears or cracks in the capsule, which causes the disc to bulge, break open (rupture), or break into fragments).

Any disc in the spine may be treated with methods of the disclosure, but in specific cases the discs are in the lower back (lumbar region) and/or the neck (cervical region).

In specific cases, the methods may employ a scaffold with one or more compositions. A scaffold used for the regeneration of biological tissue may be comprised of a material that serves as matrix to allow cells to attach to the surface of the material and form a three dimensional tissue. This material may be non-toxic, biocompatible and/or biodegradable, in specific embodiments. The most widely used biodegradable polymers, satisfying the aforementioned physical requirements, include organic polymers such as polyglycolic acid (PGA), polylactic-co-glycolic acid (PLGA), poly-c-caprolactone (PCL), polyamino acids, polyanhydrides, polyorthoesters; natural hydrogels such as collagen, hyaluronic acid, alginate, agarose, chitosan; synthetic hydrogels such as poly(ethylene oxide) (PEO), poly(vinyl alcohol) (PVA), poly(acrylic acid) (PAA), poly(propylene fumarate-co-ethylene glycol) [P(PF-co-EG) and copolymers thereof, for example.

In certain aspects, cartilage generation may begin at least in part in vitro, using autologous or allogeneic or xenogeneic nucleus pulpous cells (or a mixture thereof), chondrocytes, fibroblasts (including at least dermal fibroblasts or fibroblasts from connective tissue in the body, including bone, cartilage and/or muscle), stem cells, and/or adipocytes, for example. Any cells or mixture of cells can then be delivered to or into the joint(s) or in the vicinity of the joint(s). In embodiments wherein fibroblasts are present in the mixture, the fibroblasts may attract other non-captured fibroblasts into the cartilage regeneration process, in specific embodiments. In particular embodiments, the cells or mixture of types of cells is provided to the individual with one or more other therapeutic agents. A cellular component of the delivery may occur at the same or a different time as a non-cellular component of the delivery.

In certain embodiments, a matrix may be introduced and then seeded either in vitro or in vivo with the gene therapy and/or fibroblasts and/or chondrocytes to provide structure to the cartilage regeneration process.

Differentiation of cells into chondrocytes or chondrocyte-like cells may occur in any suitable manner, including a) differentiation of cells in vitro prior to delivery of the cells into the individual; b) differentiation of cells in vitro prior to delivery of the cells into the individual and also in vivo following delivery; and/or c) differentiation in vivo following delivery of the cells. Delivery may comprise implantation of one or more compositions of the cells. In some cases, the cells are provided to an individual with a needle-like instrument with a long tube open at the distal end.

Embodiments of the disclosure include methods of generating chondrocytes or chondrocyte-like cells for an individual, comprising the step of providing to a degenerated disc of an individual an effective amount of components from the nucleus pulposus (NP) of the same individual or another individual from the same or different species. In specific embodiments, components from the NP comprise notochordal cells, small chondrocyte-like cells, collagen fibrils, proteoglycans, and/or aggrecan. One or more therapeutic agents may also be provided to the degenerated disc of the individual, in some cases, such as one or more therapeutic agents that comprise nucleic acid, peptide, protein, small molecule, or a combination thereof. Alternatively, the one or more therapeutic agents are provided to a cell mixture in vitro prior to delivery of the cell mixture to the individual. In certain embodiments, nucleic acids, peptides, and/or polypeptides that are therapeutic agents themselves are delivered into cells that are to be provided to the individual.

In specific embodiments, the method comprises the step of providing to the individual one or more compositions comprising an effective amount of one or more of the following: a) fibroblasts; b) notochordal cells; c) Tie2+ cells; d) Tie2 gene product; e) platelet-rich plasma (PRP); f) Sox9 gene product g) transforming growth factor beta-1 (TGFB 1); h) connective tissue growth factor (CTGF); i) WNT1-inducible-signaling pathway protein 1 (WISP1), and/or j) WISP2. In at least certain cases, cells of the NP are modified ex vivo prior to providing them to the individual. Cells used in the disclosure, including fibroblasts, notochordal cells, and/or Tie2+ cells, may be modified ex vivo. In some cases, cells are exposed to hypoxia, mechanical strain, or a combination thereof prior to the providing step.

Certain cells of the disclosure are modified to produce a certain product by the cells, such as the Tie2 gene product and/or the Sox9 gene product. In a specific embodiment, a cell to be delivered to an individual comprises or is modified to express a gene product selected from the group consisting of COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4, COL6A5, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, COL26A1, COL27A1, COL28A1, Gata4, Mef2C, Tbx5, Sox5, Sox6, Sox9, FGFR2, VEGF, MMP14, forkhead, CD10, MMP13, WNT11, BAPX1, IL-1R1, IGFBP5, MMP16, BMP2, ALK1, BMP5, IGF1, MMP13, ADAMTS5, BCL10, MCOLN2, LRRC8C, PTGFR, RLF, MATN1, PDPN, TNFRSF18, ITGA10, THBS3, SCYL1BP1, KCNT2, 244533_at, ARF1, 222348_at, SLC4A5, HSPC159, RHOQ, MATN3, SULT1C2, 236289_at, BCL2L11, F1116008, KLF7, NRP2, SERPINE2, FN1, B3GNT7, ADAMTS9, ANKRD28, GALNTL2, IRAK2, SETDS, FNDC3B, B3GNT5, CYTL1, TBSP, 229221_at, PET112L, EDNRA, 1563414_at, OSMR, C1QTNF3, ZFYVE16, 225611_at, MAST4, EDIL3, 230204_at, 230895_at, HAPLN1, PDLIM4, cr5q35 SQSTM1, SCUBE3, CMAH, 236685_at, BMP6, ULBP2, LRP11, SOD2, SYNJ2, WTAP, HIG2, KIAA1718, FAM62B, UBE3C, TNFRSF10D, SLC25A37, ChGn, RB1CC1, C8orf72, EIF2C2, HAS2, TRPS1, WISP1, 235821_at, PTK2, ZCCHC7, RPS6, GLIS3, SLC28A3, 1555841_at, MGC17337, EDG2, 229242_at, ITGB1, C10orf49, YME1L1, AKR1C2, CHST3, LOXL4, SFXN3, 228910_at, CD44, FOSL1, RELA, MMP12, MMP13, MMP3, KIAA0999, ASAM, LOC399959, ETNK1, SOXS, CHST11, ATF1, SRGAP1, DSPG3, LOC338758, KIAA0701, SLC41A2, RHOF, FZD10, NUPL1, USP12, UFM1, LECT1, GPC6, ERO1L, BDKRB1, SEMA6D, LACTB, ARIH1, CSPG4, AGC1, LOC283824, VASN, WWP2, NOS2A, LOC201181, MSI2, PITPNC1, TGIF, 1552288_at, 1552289_a_at, ZNF146, RELB, MIA, ZNF160, SNXS, BMP2, RNF24, HSUP1, MATN4, BIC, RUNX1, LIF, RP4-756G23.1, RPS6KA3, TNMD, RP6-213H19.1, and a combination thereof.

Cells of the disclosure may be autologous, allogeneic, or xenogeneic in relation to the individual.

In some embodiments, a method further comprises detection of the degenerated disc, such as by measuring the level of notochord cells in the NP of a disc in the individual suspected of being degenerated. The degenerated disc may be detected structurally or non-structurally (such as by monitoring the level of cells, including notochordal cells, in the disc). Non-structural detection may comprise biochemical or molecular means. Detection of the state of the degenerated disc, or the level of notochord cells in the NP of a disc, may or may not occur prior to delivery to the disc of one or more compositions of this disclosure.

In some embodiments, there is a method of repairing and/or regenerating cartilage in a spinal disc of an individual in need thereof, comprising the steps of: a) providing fibroblasts, stem cells, adipocytes, or a combination thereof to the disc of the individual; b) providing one or more components from the nucleus pulposus (NP) to the disc of the individual; and c) providing one or more of the following to the disc of the individual: 1) Tie2+ cells; 2) Tie2 gene product; 3) platelet-rich plasma (PRP); 4) PRP+ cells; 5) Sox9 gene product; 6) Sox9+ gene product; 7) TGFB1 gene product; 8) TGFB 1+ cells; 9) CTGF gene product; 10) CTGF+ cells; 11) WISP1 gene product; 12) WISP1+ cells; 13) WISP2 gene product; and/or 14) WISP2+ cells. In specific embodiments, one or more components of the NP comprise notochordal cells, small chondrocyte-like cells, collagen fibrils, and/or aggrecan. In certain cases, step a) occurs prior to steps b) and/or c).

In another embodiment, there is a method of repairing and/or regenerating cartilage in a spinal disc of an individual in need thereof, comprising the steps of: combining one or more of the compositions listed in a), b) and/or c) with another one or more of the compositions listed in a), b), and/or c) to produce a mixture: a) fibroblasts, stem cells, adipocytes, or a combination thereof; b) one or more components from the nucleus pulposus (NP); c) one or more of the following: 1) Tie2+ cells; 2) Tie2 gene product; 3) platelet-rich plasma (PRP); 4) PRP+ cells; 5) Sox9 gene product; 6) Sox9+ cells; 7) TGFB1 gene product; 8) TGFB1+ cells; 9) CTGF gene product; 10) CTGF+ cells; 11) WISP1 gene product; 12) WISP1+ cells, 13) WISP2 gene product; and/or 14) WISP2+ cells; and providing the mixture to the individual. In specific aspects, the mixture is generated in vitro or is generated in vivo. In specific cases, the mixture is delivered to the individual by injection or by insertion via open incision.

In particular embodiments, methods of the disclosure include delivering a therapeutically effective amount of notochordal cells and/or notochordal cell conditioned medium to an individual in need thereof, including an individual with degenerative disc(s), for example. In specific embodiments, in addition to notochordal cell conditioned medium or one or more components therefrom, an individual is provided with an effective amount of notochordal cells, small chondrocyte-like cells, collagen fibrils, proteoglycans, and/or aggrecan, for example. The notochordal cell conditioned medium regenerates the degenerative disc or regenerates a portion thereof or generates chondrocytes or chondrocyte-like cells in the individual. Providing the notochordal cell conditioned medium to the individual may treat degenerative disc, reverse degenerative disc, prevent degenerative disc, or prevent further deterioration of degenerative disc. In specific embodiments, the notochordal cell conditioned medium is serum free. In specific embodiments, one or more components from notochordal cells and/or notochordal cell conditioned medium comprises transforming growth factor beta-1 (TGFB1), connective tissue growth factor (CTGF, also called CCN2), WNT1-inducible-signaling pathway protein 1 (WISP1), and/or WISP2. In some cases, in addition to or instead of delivering notochordal cells and/or notochordal cell conditioned medium to the individual, one may deliver TGFB1, CTGF, WISP1, and/or WISP2 to the individual, either in protein form or in nucleic acid form. Upon delivery of notochordal cells and/or notochordal cell conditioned medium and/or TGFB1, CTGF, WISP1, and/or WISP2, there is retention of notochordal cells and/or stem cells in the nucleus pulposus, as compared to loss of notochordal cells and/or stem cells in the absence of such a delivery.

An individual may receive multiple administrations of the therapeutic composition(s), and the separate administrations may be delivered within any span of time, such as within days, weeks, months, and/or years of another. An individual may receive administrations of multiple therapeutic composition(s) of the disclosure via different routes. In some cases, an individual yet to have one or more symptoms of a degenerated disc are provided one or more therapeutic composition(s) of the disclosure. In certain cases, an individual is provided an effective amount of one or more therapeutic composition(s) of the disclosure beginning at a certain age, such as at 20, 25, 30, 35, 40, 45, 50, 55, 60, 65, 70, 75 years, and so forth. In particular aspects, an individual prone to having a degenerated disc is provided an effective amount of one or more therapeutic composition(s) of the disclosure regardless of whether or not one or more symptoms of a degenerated disc have been detected for the individual. An individual prone to have a degenerated disc includes one that engages in repetitive activities, one having an injury to the spine, one that participates in athletics (including high-contact athletics), and/or one having a job that requires heavy lifting, for example.

In some cases, an effective amount of one or more components from the nucleus pulposus (NP) are provided to an individual in need of receiving the one or more components from the NP; in specific cases the one or more components from the NP include notochordal cells, notochordal cell conditioned media, small chondrocyte-like cells, collagen fibrils, proteoglycans, and/or aggrecan, for example. The individual may be provided a therapeutic agent also. In particular embodiments, the individual is provided one or more components from the NP for the purpose of specifically providing the one or more components from the NP to the individual. An individual may be determined to need the one or more components from the NP prior to specifically providing the one or more components from the NP to the individual. In some cases, the individual is specifically provided one or more components from the NP but is specifically not provided one or more other components from the NP. In specific cases, a substantially entire content of NP is or is not provided to an individual. In particular embodiments, an individual is provided an effective amount of a composition that comprises, consists essentially of, or consists of one or more components from the NP, such as one or more of notochordal cells, notochordal cell conditioned media, small chondrocyte-like cells, collagen fibrils, proteoglycans, and/or aggrecan, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. The present application refers to a number of references and documents all of which are incorporated herein in their entirety.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

U.S. Pat. No. 7,850,983, U.S. Patent Publication US 2014/0314726; U.S. Patent Publication US 2014/0044682; U.S. Patent Publication US 2014/0377231; and WO/2015/035395 are incorporated by reference herein in their entirety.

### I. Definitions

The term “chondrocyte-like cells” as used herein refers to cells that are not primary chondrocytes but are derived from stem cells (such as mesenchymal stem cells) or cells from other lineages (such as fibroblasts). These chondrocyte-like cells have a phenotype of chondrocytes (cells of cartilage). This means that not only do they have a shape of chondrocytes (polygonal and/or rhomboidal cells, for example), but also they are able to aggregate and produce cartilage matrix components, such as sulfated proteoglycan and type II collagen, for example. Thus, exemplary markers of chondrocyte-like cells include one or more of aggrecan, which is a chondroitin sulfate and keratan sulfate proteoglycan, type II collagen, Sox-9 protein, cartilage link protein, and perlecan, which is a heparan sulfate proteoglycan, for example.

The term “hypoxia” as used herein refers to a deficiency in oxygen. In specific aspects, it refers to oxygen tension that is less than about 20%, 15%, 10%, 5%, and so forth.

The term “joint” as used herein refers to a region in the body wherein two bones of a skeleton join.

In certain embodiments, the term “seeding” as used herein refers to implanting cells in a scaffold, and the scaffold may be present in a disc or may be present in vitro. The cells will attach to the scaffold and then grow and differentiate in the scaffold. In specific embodiments, the term “seeding” refers to seeding the cells into the nucleus pulpous via direct injection without a scaffold. This differentiation can occur both in vitro and in vivo.

### II. General Embodiments

As a rapidly expanding field, tissue engineering provides alternative solutions for articular cartilage repair and regeneration through developing biomimetic tissue substitutes, in at least some cases. In some embodiments of the disclosure, there are methods and compositions related to repair and/or regeneration of tissue, including cartilage. As used herein, the term “repair” denotes the restoration of normal function of cartilage regardless of the composition of new tissue that fills the defect sites. As used herein, “regeneration” is defined as a process that not only restores the normal functions of injured or diseased articular cartilage, but also results in the formation of new tissue that is indistinguishable from or very similar to the native cartilage.

In particular embodiments, nucleus pulposus cells and/or conditioned medium generated therefrom are provided to an individual in need thereof, such as an individual that has degenerative disc disorder, has a degenerative disc, and so forth, and it may treat or prevent herniated disc, bulging disc, slipped disc, ruptured disc, and so forth. The cells from the nucleus pulposus and/or conditioned medium generated therefrom may be obtained from the individual being treated, such as from a healthy disc from the same individual; they may be obtained from another individual of the same species; or they may be obtained from an individual of another species, for example. Methods of isolating nucleus pulposus cells are known in the art (for example, Gruber et al., 2006; Feng et al., 2013; Tang et al., 2014). The nucleus pulposus cells may be obtained commercially. Methods of generating conditioned medium from cells are known in the art.

The cells from the nucleus pulposus (and/or any other cells to be delivered to an individual), may be exposed to one or more compositions prior to delivery to an individual in need thereof. For example, the cells may be modified to express one or more compositions that are conducive to cellular health and/or proliferation and/or the cells may be modified to express one or more compositions for cartilage repair and/or regeneration. In specific embodiments, the cells are manipulated to harbor a vector (viral (adenoviral, adeno-associated, lentiviral, retroviral, and so forth) or non-viral (plasmid)) that expresses a particular nucleic acid for therapeutic purposes (wherein the nucleic acid itself is therapeutic or wherein the nucleic acid expresses a therapeutic gene product). In other embodiments, cells or other vectors (such as liposomes) are manipulated to provide a particular protein or functionally active peptide fragment thereof. Proteins may be delivered as fusion proteins, for example a protein of interest may be fused with another protein or protein fragment that facilitates cartilage repair and/or regeneration or the protein of interest may be fused with another protein or protein fragment, such as a marker or label. Examples of proteins that may be provided to the individual, or nucleic acids that encode part or all of them, are as follows: COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4, COL6A5, COL7A1, COL8A1, COL8A2, COL9A1, COL9A2, COL9A3, COL10A1, COL11A1, COL11A2, COL12A1, COL13A1,COL14A1, COL15A1, COL16A1, COL17A1, COL18A1, COL19A1, COL20A1, COL21A1, COL22A1, COL23A1, COL24A1, COL25A1, COL26A1, COL27A1, COL28A1, Gata4, Mef2C, Tbx5, Sox5, Sox6, Sox9, FGFR2, VEGF, MMP14, forkhead, CD10, MMP13, WNT11, BAPX1, IL-1R1, IGFBP5, MMP16, BMP2, ALK1, BMP5, IGF1, MMP13, ADAMTS5, BCL10, MCOLN2, LRRC8C, PTGFR, RLF, MATN1, PDPN, TNFRSF18, ITGA10, THBS3, SCYL1BP1, KCNT2, 244533_at, ARF1, 222348_at, SLC4A5, HSPC159, RHOQ, MATN3, SULT1C2, 236289_at, BCL2L11, F1116008, KLF7, NRP2, SERPINE2, FN1, B3GNT7, ADAMTS9, ANKRD28, GALNTL2, IRAK2, SETDS, FNDC3B, B3GNT5, CYTL1, TBSP, 229221_at, PET112L, EDNRA, 1563414_at, OSMR, C1QTNF3, ZFYVE16, 225611_at, MAST4, EDIL3, 230204_at, 230895_at, HAPLN1, PDLIM4, cr5q35 SQSTM1, SCUBE3, CMAH, 236685_at, BMP6, ULBP2, LRP11, SOD2, SYNJ2, WTAP, HIG2, KIAA1718, FAM62B, UBE3C, TNFRSF10D, SLC25A37, ChGn, RB1CC1, C8orf72, EIF2C2, HAS2, TRPS1, WISP1, 235821_at, PTK2, ZCCHC7, RPS6, GLIS3, SLC28A3, 1555841_at, MGC17337, EDG2, 229242_at, ITGB1, C10orf49, YME1L1, AKR1C2, CHST3, LOXL4, SFXN3, 228910_at, CD44, FOSL1, RELA, MMP12, MMP13, MMP3, KIAA0999, ASAM, LOC399959, ETNK1, SOXS, CHST11, ATF1, SRGAP1, DSPG3, LOC338758, KIAA0701, SLC41A2, RHOF, FZD10, NUPL1, USP12, UFM1, LECT1, GPC6, ERO1L, BDKRB1, SEMA6D, LACTB, ARIH1, CSPG4, AGC1, LOC283824, VASN, WWP2, NOS2A, LOC201181, MSI2, PITPNC1, TGIF, 1552288_at, 1552289_a_at, ZNF146, RELB, MIA, ZNF160, SNXS, BMP2, RNF24, HSUP1, MATN4, BIC, RUNX1, LIF, RP4-756G23.1, RPS6KA3, TNMD, RP6-213H19.1, osmosensitive transcription factor TonEBP and a combination thereof. The aforementioned gene products may be delivered as nucleic acid or as protein.

In other embodiments, any cells to be delivered to an individual in need thereof may be exposed to one or more conditions that provide, facilitate, or enhance therapy for the individual, such as provide, facilitate, or enhance cartilage regeneration and/or repair. In specific embodiments, any cells of the disclosure may be exposed to hypoxia conditions (such as low oxygen tension, for example 5% or less O₂), hyperosmotic environment, mechanical strain, or a combination thereof, prior to delivery to the individual, and in other embodiments the cells are alternatively or additionally exposed to hypoxia conditions, mechanical strain, or a combination thereof following in vivo delivery. Examples of mechanical strain include intermittent hydrostatic pressure, fluid shear stress, low oxygen tension, direct compression, or a combination thereof.

In specific embodiments, Tie2+ cells are provided to an individual in need thereof. Tie2 may also be referred to as Angiopoietin-1 receptor; CD202B; hTIE2; p140 TEK; soluble TIE2 variant 1; soluble TIE2 variant 2; TEK; TEK tyrosine kinase, endothelial; TIE-2; TIE2; Tunica interna endothelial cell kinase; Tyrosine-protein kinase receptor TEK; Tyrosine-protein kinase receptor TIE-2; VMCM; and VMCM1. Tie2+cells may be produced, such as by transforming or transfecting a cell in question with a vector comprising a nucleic acid that encodes part or all of the Tie2 protein. Tie2+ cells may be isolated from the individual being treated or from another individual, including another individual of the same or different species. Tie2+ cells may be isolated using an entity that binds Tie2+, such as a Tie2 antibody or aptamer, for example, and these methods are well known in the art.

Cells from the nucleus pulposus and/or conditioned medium generated therefrom are provided to the individual, in certain embodiments, and such cells may comprise notochordal cells, small chondrocyte-like cells, or a combination thereof. In addition to cell delivery, other components from the nucleus pulposus may be provided to the individual, such as collagen fibrils and/or aggrecan or any proteoglycan. Any compositions for delivery may be labeled, and compositions may also include antibiotic(s), buffers, salts, media, and so forth.

In particular aspects, methods of the disclosure include the step of identification of a joint medical condition or disorder or defect, including the joint being a spinal disc. Methods of determining disc defects are known in the art, but in specific embodiments they include CT scan, magnetic resonance imaging, discogram, a combination thereof, and so forth. In certain cases, when it is determined that a disc has a reduction in notochordal cells, the individual may be in need of therapy of the disclosure.

Individuals for treatment may have disc issues with unknown cause, or the individuals may be 50 or older than 50, or athletes, for example. In specific embodiments, the individual is in their 20s, 30s, or 40s.

In particular embodiments, the disclosure encompasses methods for improving the environment of an aged disc by providing a cell blend that comprises a content of cells and numbers of cells that are present or isolated from one or more younger, more virile discs. Methods are included for improving the condition of an aged disc in an individual by providing to the individual with the aged disc an effective amount of a cell blend from, or that one would be found in, a disc of a person without the onset of the degenerative process. In particular embodiments, the cell blend comprises fibroblasts; stem cells; adipocytes; notochordal cells; Tie2+ cells; PRP+ cells; Sox9+ cells; TGFB1+ cells; CTGF+ cells; WISP1+ cells, WISP2+ cells, or a combination thereof. In certain cases, the individual is provided one or more of the following: Tie2 gene product; platelet-rich plasma (PRP); Sox9 gene product; TGFB1 gene product; CTGF gene product; WISP1 gene product; WISP2 gene product; or a combination thereof.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

As an example of a method of the disclosure, there is introduction of allogeneic, xenogenic or autologous nucleus pulposus cells and/or conditioned medium generated therefrom, and/or Sox9 or other genes that aid in the production of cartilage and/or notochordal cells, and/or Tie2+ cells, and/or platelet-rich plasma (PRP). Such delivery will benefit the differentiation process by delivering cells found in abundance in healthy nucleus pulposus, in certain embodiments. The reduction in quantities of Sox9 or other genes, notochordal cells, and/or Tie2+ play an important role in the onset of degenerative disc disease. Adding these cells back to a new nucleus created from a fibroblast differentiation, will result in a more robust and vibrant environment for the newly differentiated cells. Because cartilage typically has low blood flow and access to nutrients, it is considered the most difficult tissue in the body to regenerate. For this reason, other elements may be added to the fibroblast mixture such Sox9 or other genes, notochordal cells, Tie2+ cells, and/or PRP. Current research suggests the reduction in these cell types may actually be the catalyst or genesis of degenerative disc disease.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PATENTS AND PATENT APPLICATIONS

U.S. Pat. No. 7,850,983, U.S. Patent Publication US 2014/0314726; U.S. Patent Publication US 2014/0044682; U.S. Patent Publication US 2014/0377231; and WO/2015/035395

### PUBLICATIONS

Feng, G., L. Li, H. Liu, Y. Song, F. Huang, C. Tu, B. Shen, Q. Gong, T. Li, L. Liu, J. Zeng, Q. Kong, M. Yi, M. Gupte, P. X. Ma, F. Pei; Hypoxia differentially regulates human nucleus pulposus and annulus fibrosus cell extracellular matrix production in 3D scaffolds. Osteoarthritis and Cartilage 21 (2013) 582-588

Gruber HE1, Hoelscher G L, Leslie K, Ingram J A, Hanley E N Jr. Three-dimensional culture of human disc cells within agarose or a collagen sponge: assessment of proteoglycan production. Biomaterials. 2006 Jan;27(3):371-6. Epub 2005 Aug 11.

Lijie Zhang, Jerry Hu, Kyriacos A. Athanasiou The Role of Tissue Engineering in Articular Cartilage Repair and Regeneration Crit Rev Biomed Eng. 2009; 37(1-2): 1-57.

Majumdar MK, Wang E and Morris EA. BMP-2 and BMP-9 promotes chondrogenic differentiation of human multipotential mesenchymal cells and overcomes the inhibitory effect of IL-1. J. Cell. Physiol. 2001(189):275-284.

Seppa, N. Cartilage creation. New joint tissue could keep people moving, reducing need for knee or hip replacements. Science News 2012 (189 #3):22.

Tang, X., William J. Richardson, Robert D. Fitch, Christopher R. Brown, Robert E. Isaacs, A new non-enzymatic method for isolating human intervertebral disc cells preserves the phenotype of nucleus pulposus cells Jun ChenCytotechnology (2014) 66:979-986.

Wang SZ1, Rui Y F, Lu J, Wang C. Cell and molecular biology of intervertebral disc degeneration: current understanding and implications for potential therapeutic strategies. Cell Prolif. 2014 Oct;47(5):381-90. doi: 10.1111/cpr.12121. Epub 2014 Aug 11.

Zaslav K. Cole B. et al: The American Journal of Sports Medicine. 2009;37(1):42-55.

Zaslav K. Cole B. et al. A Prospective Study of Autologous Chondrocyte Implantation in Patients Who Failed Prior Treatments for Articular Cartilage The American Journal of Sports Medicine. 2009;37(1):42-55.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

I claim:

1. A method of generating chondrocytes or chondrocyte-like cells for an individual, comprising the step of providing to a degenerated disc of an individual an effective amount of one or more components from the nucleus pulposus (NP) of the same individual or another individual from the same or different species.

2. The method of claim 1, wherein components from the NP comprise notochordal cells, notochordal cell conditioned media, small chondrocyte-like cells, collagen fibrils, proteoglycans, and/or aggrecan.

3. The method of claim 1 or 2, wherein one or more therapeutic agents are provided to the degenerated disc of the individual.

4. The method of claim 1, 2, or 3, wherein the one or more therapeutic agents comprise nucleic acid, peptide, protein, small molecule, or a combination thereof.

5. The method of any one of claims 1-4, comprising the step of providing to the individual one or more compositions comprising an effective amount of one or more of the following:
a) fibroblasts;
b) notochordal cells;
c) Tie2+ cells;
d) Tie2 gene product;
e) platelet-rich plasma (PRP);
f) Sox9 gene product;
g) transforming growth factor beta-1 (TGFB 1);
h) connective tissue growth factor (CTGF);
i) WNT1-inducible-signaling pathway protein 1 (WISP1), and/or
j) WISP2.

6. The method of any one of claims 1-5, wherein cells of the NP are modified ex vivo prior to providing them to the individual.

7. The method of claim 5, wherein the fibroblasts, notochordal cells, and/or Tie2+ cells are modified ex vivo.

8. The method of any one of claims 1-7, wherein cells are exposed to hypoxia, mechanical strain, or a combination thereof prior to the providing step.

9. The method of claim 8, wherein the mechanical strain comprises intermittent hydrostatic pressure, fluid shear stress, low oxygen tension, direct compression, or a combination thereof.

10. The method of any one of claims 1-9, wherein the Tie2 gene product is produced by one or more of the cells.

11. The method of any one of claims 1-9, wherein the Sox9 gene product is produced by one or more of the cells.

12. The method of claim 3 or 4, wherein the agent comprises a gene product selected from the group consisting of COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4, COL6A5, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, COL26A1, COL27A1, COL28A1, Gata4, Mef2C, Tbx5, Sox5, Sox6, Sox9, FGFR2, VEGF, MMP14, forkhead, CD10, MMP13, WNT11, BAPX1, IL-1R1, IGFBP5, MMP16, BMP2, ALK1, BMPS, IGF1, MMP13, ADAMTSS, BCL10, MCOLN2, LRRC8C, PTGFR, RLF, MATN1, PDPN, TNFRSF18, ITGA10, THBS3, SCYL1BP1, KCNT2, 244533_at, ARF1, 222348_at, SLC4A5, HSPC159, RHOQ, MATN3, SULT1C2, 236289_at, BCL2L11, F1116008, KLF7, NRP2, SERPINE2, FN1, B3GNT7, ADAMTS9, ANKRD28, GALNTL2, IRAK2, SETDS, FNDC3B, B3GNT5, CYTL1, TBSP, 229221_at, PET112L, EDNRA, 1563414_at, OSMR, C1QTNF3, ZFYVE16, 225611_at, MAST4, EDIL3, 230204_at, 230895_at, HAPLN1, PDLIM4, cr5q35 SQSTM1, SCUBE3, CMAH, 236685_at, BMP6, ULBP2, LRP11, SOD2, SYNJ2, WTAP, HIG2, KIAA1718, FAM62B, UBE3C, TNFRSF10D, SLC25A37, ChGn, RB1CC1, C8orf72, EIF2C2, HAS2, TRPS1, WISP1, 235821_at, PTK2, ZCCHC7, RPS6, GLIS3, SLC28A3, 1555841_at, MGC17337, EDG2, 229242_at, ITGB1, C10orf49, YME1L1, AKR1C2, CHST3, LOXL4, SFXN3, 228910_at, CD44, FOSL1, RELA, MMP12, MMP13, MMP3, KIAA0999, ASAM, LOC399959, ETNK1, SOXS, CHST11, ATF1, SRGAP1, DSPG3, LOC338758, KIAA0701, SLC41A2, RHOF, FZD10, NUPL1, USP12, UFM1, LECT1, GPC6, ERO1L, BDKRB1, SEMA6D, LACTB, ARIH1, CSPG4, AGC1, LOC283824, VASN, WWP2, NOS2A, LOC201181, MSI2, PITPNC1, TGIF, 1552288_at, 1552289_a_at, ZNF146, RELB, MIA, ZNF160, SNXS, BMP2, RNF24, HSUP1, MATN4, BIC, RUNX1, LIF, RP4-756G23.1, RPS6KA3, TNMD, RP6-213H19.1, and a combination thereof.

13. The method of any one of claims 1-12, wherein the cells are autologous, allogeneic, or xenogeneic in relation to the individual.

14. The method of any one of claims 1-13, wherein the method further comprises detection of the degenerated disc.

15. The method of claim 14, wherein the degenerated disc is detected by measuring the level of notochord cells in the NP of a disc in the individual suspected of being degenerated.

16. The method of claim 14, wherein the degenerated disc is detected structurally or non-structurally.

17. The method of claim 16, wherein non-structural detection comprises by biochemical or molecular means.

18. The method of any one of claims 1-17, wherein the individual is also provided an effective amount of fibroblasts, stem cells, and/or adipocytes.

19. A method of repairing and/or regenerating cartilage in a spinal disc of an individual in need thereof, comprising the steps of:
a) providing fibroblasts, stem cells, adipocytes, or a combination thereof to the disc of the individual;
b) providing one or more components from the nucleus pulposus (NP) to the disc of the individual; and
c) providing one or more of the following to the disc of the individual:
1) Tie2+ cells;
2) Tie2 gene product;
3) platelet-rich plasma (PRP);
4) PRP+ cells;
5) Sox9 gene product;
6) Sox9+ cells
7) TGFB1 gene product;
8) TGFB1+ cells;
9) CTGF gene product;
10) CTGF+ cells;
11) WISP1 gene product;
12) WISP1+ cells,
13) WISP2 gene product; and/or
14) WISP2+ cells.

20. The method of claim 19, wherein the one or more components of the NP comprise notochordal cells, small chondrocyte-like cells, collagen fibrils, and/or aggrecan.

21. The method of claim 19 or 20, wherein step a) occurs prior to steps b) and/or c).

22. The method of any one of claims 19-21, wherein the individual is also provided an effective amount of fibroblasts, stem cells, and/or adipocytes.

23. A method of repairing and/or regenerating cartilage in a spinal disc of an individual in need thereof, comprising the steps of:
combining one or more of the compositions listed in a), b) and/or c) with another one or more of the compositions listed in a), b), and/or c) to produce a mixture:
a) fibroblasts, stem cells, adipocytes, or a combination thereof;
b) one or more components from the nucleus pulposus (NP);
c) one or more of the following:
1) Tie2+ cells;
2) Tie2 gene product;
3) platelet-rich plasma (PRP);
4) PRP+ cells;
5) Sox9 gene product; and
6) Sox9+ cells;
7) TGFB1 gene product;
8) TGFB1+ cells;
9) CTGF gene product;
10) CTGF+ cells;
11) WISP1 gene product;
12) WISP1+ cells,
13) WISP2 gene product; and/or
14) WISP2+ cells; and
providing the mixture to the individual.

24. The method of claim 23, wherein the mixture is generated in vitro.

25. The method of claim 23, wherein the mixture is generated in vivo.

26. The method of any one of claims 23-25, wherein the mixture is delivered to the individual by injection, by insertion via open incision, by catheter, or a combination thereof.

27. The method of any one of claims 23-26, wherein the individual is also provided an effective amount of fibroblasts, stem cells, and/or adipocytes.

28. A method of improving the condition of an aged disc in an individual by providing to the individual with the aged disc an effective amount of a cell blend from, or that one would be found in, a disc of a person without the onset of the degenerative process.

29. The method of claim 28, wherein the cell blend comprises fibroblasts; stem cells; adipocytes; notochordal cells; Tie2+ cells; PRP+ cells; Sox9+ cells; TGFB1+ cells; CTGF+ cells; WISP1+ cells, WISP2+ cells, or a combination thereof.

30. The method of claim 28 or 29, wherein the individual is provided one or more of the following: Tie2 gene product; platelet-rich plasma (PRP); Sox9 gene product; TGFB1 gene product; CTGF gene product; WISP1 gene product; WISP2 gene product; or a combination thereof.
